(19) United States
(12) Patent Application Publication
(10) Pub. No.: US 2017/0246026 A1
(43) Pub. Date: Aug. 31, 2017

(54) A HUMAN EXTERNAL URINARY INCONTINENCE TREATMENT METHOD AND DEVICE

(71) Applicant: GR Dome Medical Ltd., Tirat Carmel (IL)

(72) Inventor: Amir LANIADO, Haifa (IL)

(21) Appl. No.: 15/302,990

(22) PCT Filed: Aug. 14, 2014
(86) PCT No.: PCT/IL14/00039

§ 371 Date: Oct. 9, 2016

Related U.S. Application Data
(60) Provisional application No. 61/988,219, filed on May 4, 2014.

(51) Int. Cl. A61F 5/443; A61F 5/453; A61F 5/455; A61F 5/44

(52) U.S. Cl. CPC A61F 5/443; A61F 5/4408; A61F 5/453; A61F 5/455

(57) ABSTRACT

The present invention relates to an external urinary incontinence treatment a device and a method of deploying an external urinary incontinence treatment device that minimizes the inconvenience of fastening and fixating a urine receiving component to the genital region and to the skin surrounding the urethral orifice of a treated patient. The device comprises: a urine receiving component, a tube, a receiving component supporting element, a tube locking system, a genital region connection component, and a genital region anchoring element. The tube connects and communicates freely with the urine receiving component and is inserted through the receiving component supporting element. The tube also communicates with said tube locking system and is able to move vertically the receiving component supporting element when said tube locking system is deactivated. The tube is fixated in its movement at a desired position along the length of said tube when said tube locking system is activated. The device receiving component supporting element is connected to the genital region connection component and with the tube locking system. The genital region connection component is connected to the genital region anchoring element. In deployment of the device, the genital region anchoring element is reversibly connected to the skin of the genital region of the treated patient, and urine receiving-component is connected to the skin surrounding the urethral orifice of the treated patient when the tube is moved towards the genital region of the patient. The tube is reversibly fixated in place by the tube locking system after urine receiving component is adjusted and fastened to the skin surrounding the urethral orifice of the patient in a urine leak free connection while applying the minimal required pressure, thus, causing the treated patient minimal inconvenience. The tube lucking system can be easily deactivated and reactivated to adjust the connection of the receiving component to the skin surrounding the urethral orifice in accordance to changing body postures of the patient.

### FIELD OF THE INVENTION

The present invention relates to an urinary incontinence treatment method and device for a human patient. More specifically, the present invention relates to a method of deploying a human external urinary incontinence treatment device human and an external urinary incontinence treatment device and that minimizes the inconvenience of fastening and fixating a urine receiving component to the genital region and to the skin surrounding the urethral orifice of a treated patient.

### BACKGROUND OF THE INVENTION

Various devices for the treatment of urinary incontinence are well known from medicinal publications. Publications describing external incontinence treatment devices typically describe a device having a component that directly receives the urine from the urethral orifice without penetrating the orifice (referred from hereafter as to the “the urine receiving component” or “receiving component”). The urine receiving component leads the urine with minimal leaks to a tube which leads it to a collection bag. In many urinary incontinence treatment cases, external urine-receiving components are preferred over the use of an invasive catheter in the urethra, thus, reducing the pain and inconvenience associated with invasive catheter devices and reducing the incidences of contaminations caused by the entry of harmful microorganisms through the catheter.

The ability of external urinary incontinence treatment devices to collect urine with minimal spills to the surrounding while causing the user minimal discomfort relates to the spatial configuration of the urine receiving component and to the manner in which the receiving component is reversibly connected, adjusted, fastened and fixated in the genital region, near and/or over the orifice of the urethra. The term “adjusted” in context of the receiving component of the present invention refers to minor placement movements of the component to comfortably-connect to the skin in the near of the orifice of the urethra. The skin in the near of the orifice of the urethra refers to the skin that surrounds the vicinity of the orifice. The term “fastened” in context of the receiving component of the present invention, is used to describe the tightening of the receiving component to the skin surface in the near of the orifice of the urethra of a treated patient in manner that produces a reversible urine leak-free connection.

The terms “distal” and “proximal” in reference to the external urinary incontinence treatment device of the present invention relate to the relative position of components of the device as being far from and close to the genital region of a treated patient, respectively.

In the context of the present invention, in human males the genital region refers to the penis and includes the glans penis, in human females genital region refers to the internal and external region of the labia majora and labia minora as well as to the adjacent skin-area of the thighs and lower belly area in close external vicinity of the labia majora.

In the present invention, the connecting, adjusting, fastening and fixating, of the urine receiving component to the skin that surrounds the urethral orifice of a treated patient, is easily and reversibly adjusted by a mechanism that enables precisely controlled movements of the urine receiving component to and from the skin surface. The mechanism enables the fixating of the urine receiving component in placement so that the pressure applied by the urine receiving component in the contact with the skin is the minimal required contact pressure to prevent urine leakage. The term “pressure” in context of the present invention can be interchanged with the term of a force applied on a surface.

Some currently used human male external urinary incontinence treatment devices have a condom-type tube structure that tightly wraps around the penis and has an opening at their tip. The urine is discharged into the top of the condom structure (“the receiving component”). A tube leads from the opening to a collection bag. Typically, condom-type external urinary incontinence treatment devices do not stay connected for extended time periods of long hours and full days and during their time of being connected they cause inconvenience to the user after a relative short time period. Even during the relatively short time of connection, the urine that remains “entrapped” in the condom volume is potentially susceptible to cause microbiological contaminations of the skin and urinary tract.

An example of a condom-type external urinary incontinence treatment devices is given in NL8,602,986 (Wilhelmus). The patent describes a condom-like structure that is secured to the penis by an adhesive tape that is wrapped around the base of the condom and has a one way valve to reduce the backpressure and moisture in the volume enclosed by the condom connected to the penis. In addition to the inconvenience and skin-irritation caused by wearing a condom structure, the use of the one-way valve still leaves at the tip of the condom, between the urethral orifices and the valve, a substantial volume of urine that may cause contaminations.

Another approach to the construction of an external male urinary incontinence treatment device is described in U.S. Pat. No. 5,263,947 (Kay) in which a receiving component (referred in Kay as “housing”), having an outlet conduit, is connected to a plurality of leaves made of a thin film material. The receiving component is provided with an inner side disk barrier layer which seals the surface surrounding the urethral orifice and the leaves each have an adhesive side that connects to the immediate surroundings of the urethral orifice (referred in Kay as “para-meatal surface”). The disk reduces the risk of urinary tract infection and the leaves hold the receiving component over the urethral orifice so as the orifice communicates freely with entrance to the conduit in the receiving component The use of (only) sticky thin-film leave material in positioning, adjusting, fastening and fixating the housing over the urethral orifice does not provide the ability to easily and regularly adjust and fixate the receiving component around the urethral orifice over a time period of hours and days, so as to provide minimal discomfort to a treated patient.

An example of reversibly connecting a receiving component of an external urinary incontinence treatment device to genital region of women is described in UK patent application GB2,015,347 (inventors: Steer et al.). Steer et al. describes a receiving component in the form of a pad of surgical adhesive material, which is connected to a funnel and a pipe. The pipe may include a non-return valve to prevent backflow of urine. The pad may have a ridge-like projection centrally thereon constructed to extend between the labia majora of the wearer. The surgical adhesive material connects and fixates the device to the body of the patient. No reference is made to easily and regularly adjusting, fastening and fixating the external urinary incontinence treatment device during prolonged usage periods so as to cause minimal discomfort to a treated patient

Another example of connecting and fastening of an external urinary incontinence treatment device to the genital region of women is given in U.S. Pat. No. 5,263,947 (Kay) which describes a device which comprises a receiving-component (referred to as: “housing” in Kay's text) having an outlet conduit, a plurality of leaves made of a thin, vapor permeable film connected to the housing, a vapor permeable adhesive layer on the interior surface of the leaves, a microbial-barrier layer provided on an inner surface of the housing and a barrier disc provided on the inner surface of the housing. The reversible connection and fixation to genital region of women is done by the receiving-component around the urethral orifice and applying the adhesive layer to the skin surface in a wrinkle-free manner. In addition, retaining straps are connected to the housing of the device and are worn and fastened to the lower torso of the patient to stabilize the housing position. The use of adhesive layer connections with no additional ability of regularly adjusting, fastening and fixating of the housing to the surrounding of the urethral orifice without reconnecting the adhesive layer, will cause the treated patient inconvenience when in use for extended time periods. In addition, the straps are cumbersome to ware and adjust, especially in a lying down position.

An external urinary incontinence treatment method and device of the present invention, facilitates the reversible connection of a receiving component to the skin that surrounds the urethral orifice, in the treatment of both human males and females in a manner that prevents urine leakage while enabling easy and regular adjusting, fastening and fixating the placement of the receiving component. The adjusting, fastening and fixating can be done either by a care-giver or personally by the treated patient and in accordance with the change in body posture. The changing of the body posture of a treated patient refers to, but not limited to, different laying and sitting postures. The ability to easily adjust, fasten and fixate the urine receiving component minimizes the inconvenience typically associated with the use of available external urinary incontinence treatment devices. In addition, the connection of the receiving component to the skin that surrounds the urethral orifice in accordance with the present invention minimizes the amount of urine that “escapes” to the surrounding, thus minimizes the contaminations cause to the treated patient.

### SUMMARY OF THE INVENTION

The human external urinary incontinence treatment device and method of the present invention comprises: a urine receiving-component, a tube, a receiving component supporting element (also referred from herein as the: RC supporting element), a tube locking system, a genital region connection component and a genital region anchoring element. The tube locking system is interchangeably referred to as the “tube lucking mechanism”. Alternatively, the urine receiving component and the tube form a single entity. Alternatively, the locking system and the RC supporting element form a single entity. Alternatively, the urine receiving component, the tube and the tube locking system have a single entity component.

The tube is connected to and communicates freely with, the urine receiving-component. The tube is inserted through the RC supporting element and communicates with the tube locking system. The tube locking system is connected to, or alternatively, is integrated with, the, RC supporting element. The tube is able to moves in the vertical direction, towards and from the urethral orifice, in the RC supporting element when not restricted in its movement by the tube locking system. The tube locking system reversibly stops the motion ability of the tube and locks the tube from moving from a position along the length of the tube, as desired by the person deploying the device. The stopping of the motion of the tube is referred from hereafter as “fixating” the position of the tube. When the tube is fixated by the locking system, the locking system is referred to as being “activated”, thus, when the tube is able to move the locking system is “deactivated”. In embodiments of the present invention, three tube locking systems are described: a clip-lock system, a ratchet system and a screw system. The tube locking system of the present invention is not limited to any of the three described mechanisms and can be in other mechanical configurations. RC supporting element is connected to the genital region connection component, and the genital region connection component connects to the genital region anchoring element. Optionally, both the genital region connection component and the genital region anchoring element are each composed of two or more sub components and elements, respectively. The genital region anchoring element reversibly connects to the body of a treated patient. The term “anchored” refers in the text to having a firm and stable yet reversible connection between the genital region connection component and the skin of the genital region of the treated patient. When the external urinary incontinence treatment device of the present invention is deployed, the RC supporting element connects to genital region, and provides a “fixated platform” from which (pushing) pressure is applied by the urine receiving component to the skin around the urethral orifices via the vertical proximal-directed motion and locking in place of the tube. The term: “fixated platform” refers to the RC supporting element being at a stable and fixated positioned above the urethral orifice. The applied pressure is produced by increasing the tube's distance between the receiving-component and the RC supporting element which does not change its spatial placing with the movement of the tube. When applying (pushing) pressure on the urine receiving component, the motion of the tube also applies (pulling) pressure to the genital region of the treated patient by the genital region connection component since the genital region connection component is connected to the RC supporting element. The pressure is applied by the genital connection component to the genital region of the treated patient via the genital region anchoring element.

The connecting and fastening of the urine receiving-component to the skin around the urethral orifice of the patient is separate and independent of the connecting of the RC-supporting-element to the genital region of the treated patient.

Typically, the genital region anchoring element is a thin adhesive film that connects to the distal side of the genital region connection component and reversibly connects, by a sticky surface side, to the genital region of the treated patient. Optionally, the distal (sticky) side of the thin adhesive film of the genital region anchoring element (or alternatively, sub-elements) is reversibly connected and covered by a film protection component. The film protection component maintains the stickiness of the thin adhesive film when the external urinary incontinence treatment device of the present invention is in storage. When the device is deployed, the film protection component is peeled off and discarded.

Alternatively to a thin adhesive film element or elements, the genital region connection component is connected to the body of the treated patient by a genital region anchoring element that comprises at least one “structural element”. The term structural element in the context of, the genital region connection component can be, but is not limited to, an element or elements having a configuration a of: strap, string, wire, and spring. The anchoring can be done by reversibly connecting to any location in the body of the treated patient. The term “anchoring to the body” refers to the body-regions in the near of the genital region such as, but not limited to, thighs, buttocks and hips as well as to body-regions that are within the genital region such as, but not limited to the penis, the glans penis, and the labia majora and labia minora.

In the course of deploying an external urinary incontinence treatment device of the present invention, the genital region anchoring element is reversibly connected to the body of the treated patient, thus, reversibly connecting the genital region connection component to the treated patient. In following, the urine receiving component is reversibly connected to the skin surface around the urethral orifice of the treated patient by vertically moving the tube in the RC supporting element. The tube is fixated by the tube locking system in a position along the tube in which the urine receiving component is connected to the skin surface around the urethral orifice in a urine leak-free connection while causing the treated patient minimal inconvenience.

The pressure applied to the receiving component can be easily changed and regulated in accordance with the changing of the body posture of the treated patient by deactivating the tube locking system, changing the position of the tube, thus the changing of the connection configuration of the receiving component with the skin surface around the urethral orifice, and reactivating the locking system.

### BRIEF DESCRIPTION OF THE DRAWINGS

In order to better understand the present invention, and appreciate its practical applications, the following figures are provided and referenced hereafter. It should be noted that the figures are given as examples only and in no way limit the scope of the invention. Like components are denoted by like reference numerals.

FIG. 1 to FIG. 4 illustrate and embodiment of an external urinary incontinence treatment device of the present invention, having a tube clip-locking system.

FIG. 5 to FIG. 8 illustrate a second embodiment of an external urinary incontinence treatment device of the present invention, having a tube ratchet-locking system.

FIG. 9 to FIG. 12 illustrate a third embodiment of an external urinary incontinence treatment device of the present invention, having a tube screw-locking system.

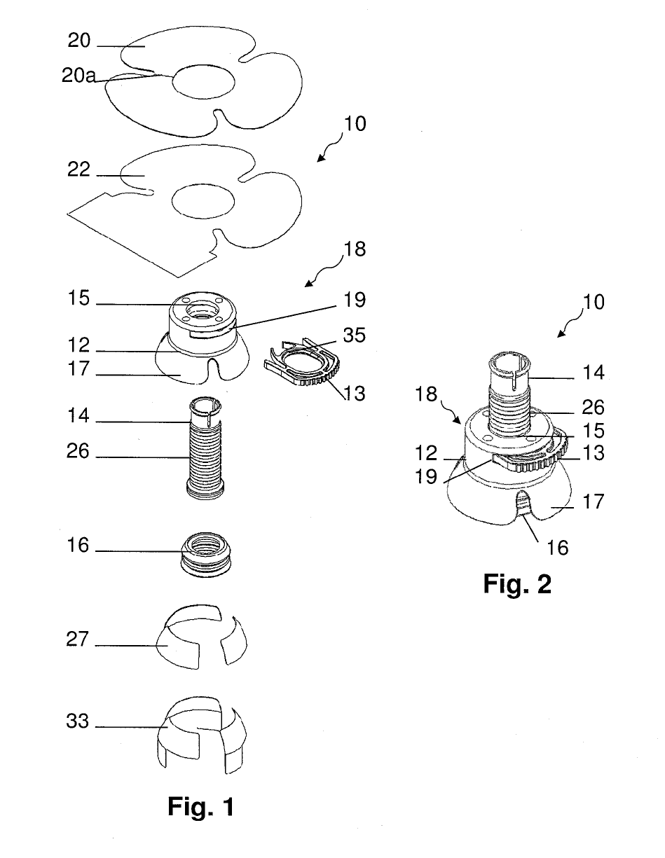

FIG. 1 is an isometric from-above-and-side view of an illustration of an assembly of components that construct an embodiment of an external urinary incontinence treatment device of the present invention having a tube clip-locking system, in a configuration for the treatment of men.

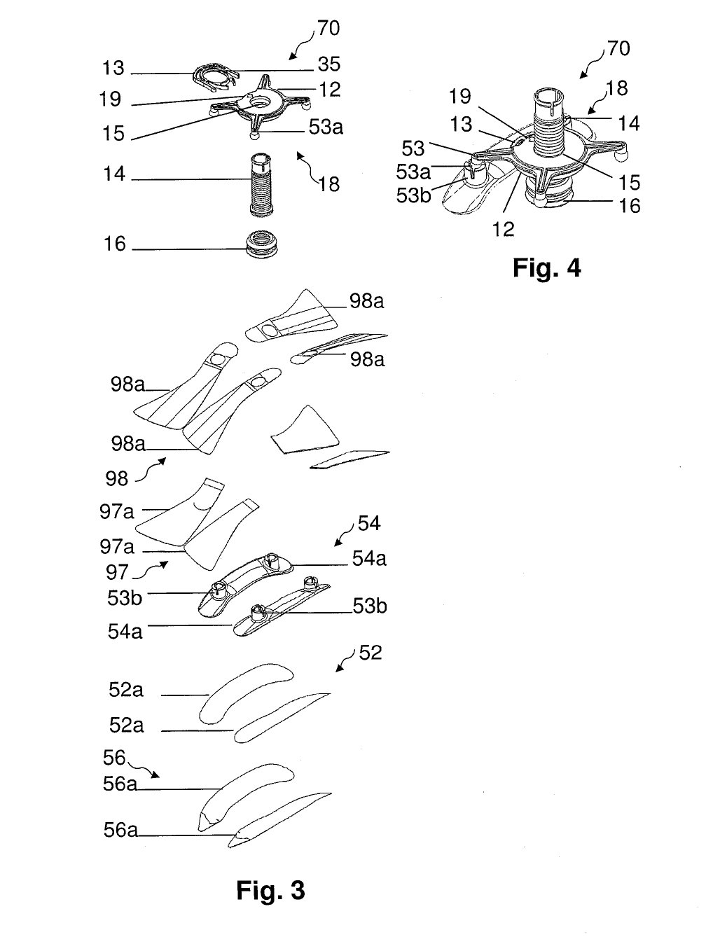

FIG. 2 is an isometric from-above-and-side view of an illustration of the embodiment of the external urinary incontinence treatment device shown in FIG. 1, in an assembled configuration.

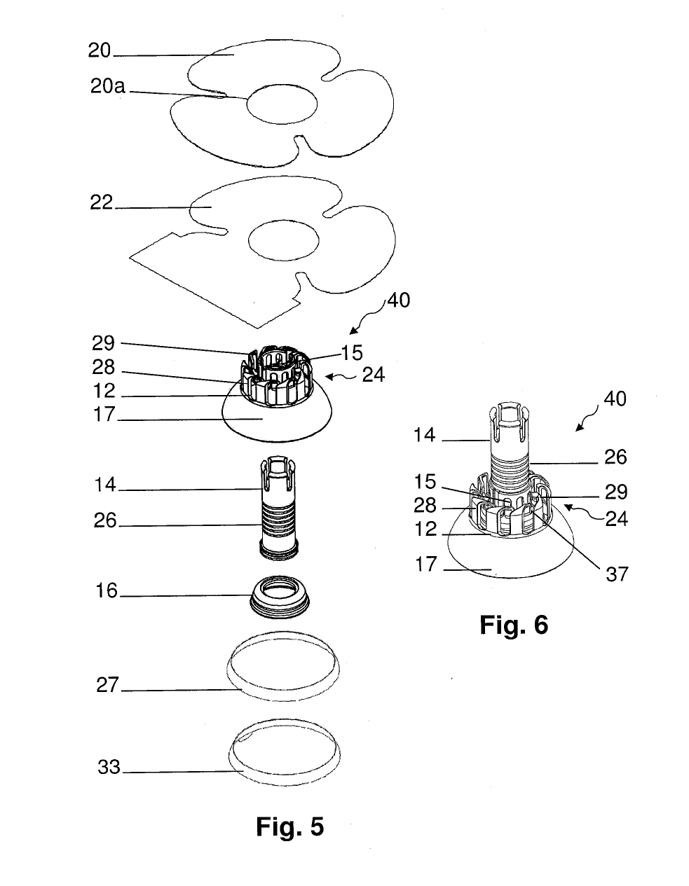

FIG. 3 is an isometric from-above-and-side view of an illustration of an assembly of components that construct an embodiment of an external urinary incontinence treatment device of the present invention having a tube clip-locking system, in a configuration for the treatment of women.

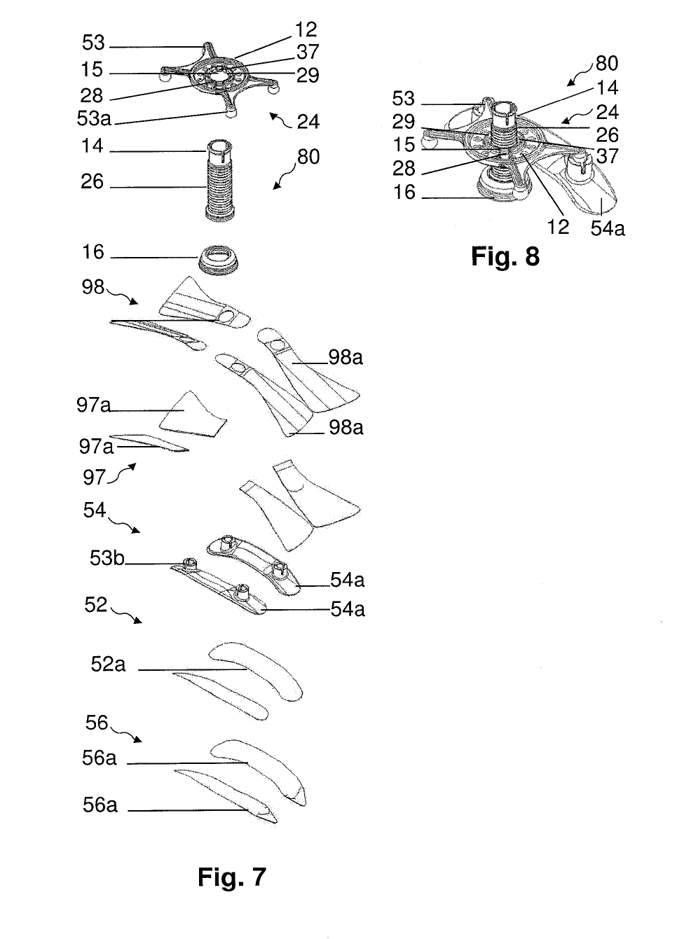

FIG. 4 is an isometric from-above-and-side view of an illustration of the embodiment of the external urinary incontinence treatment device shown in FIG. 3, in a partial assembled configuration.

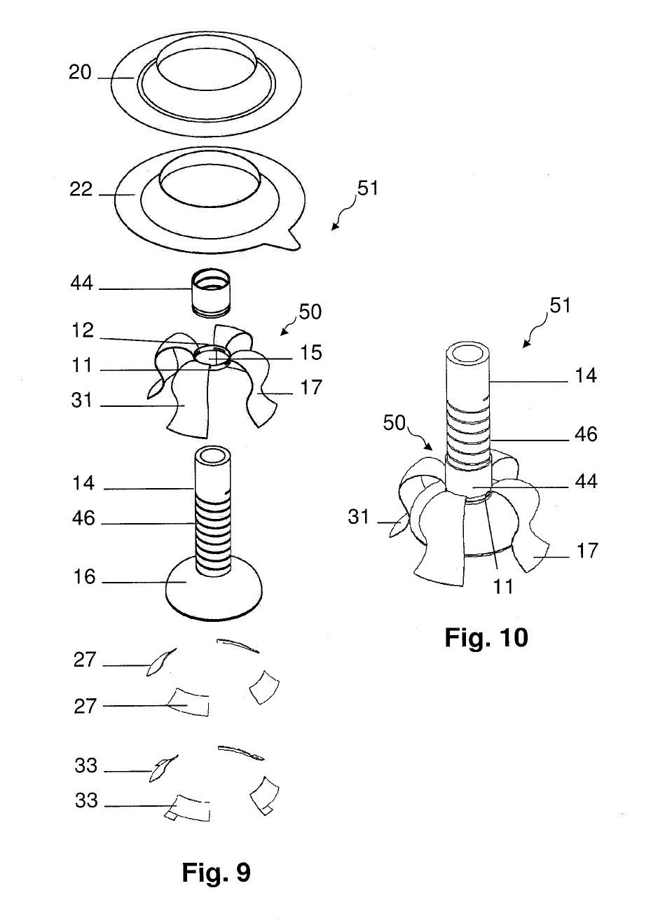

FIG. 5 is an isometric from-above-and-side view of an illustration of an assembly of components that construct a second embodiment of an external urinary incontinence treatment device of the present invention having a tube ratchet-locking system, in a configuration for the treatment of men.

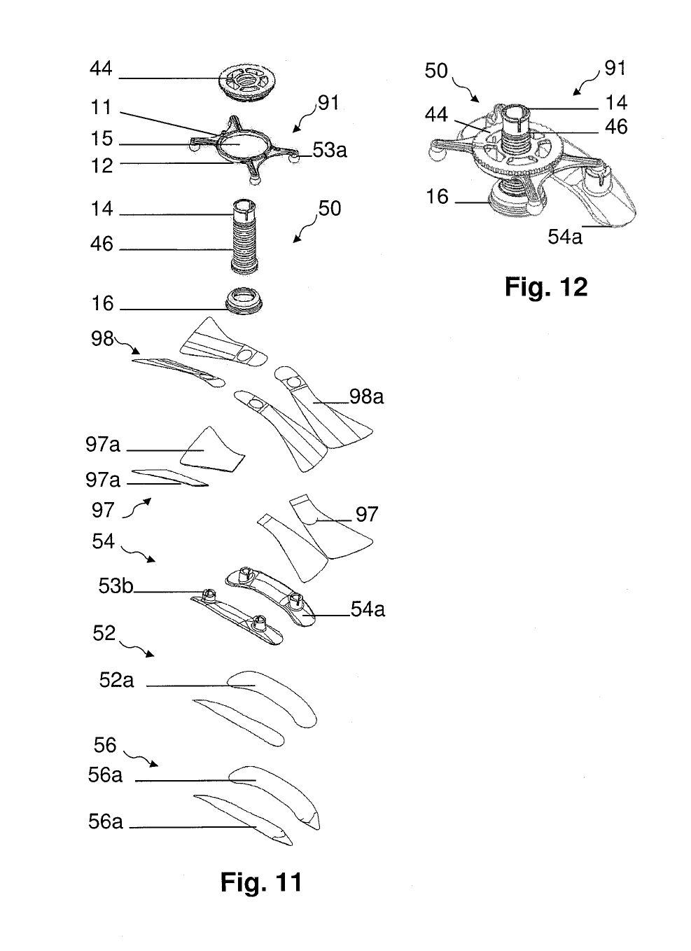

FIG. 6 is an isometric from-above-and-side view of an illustration of the embodiment of the external urinary incontinence treatment device shown in FIG. 5, in an assembled configuration.

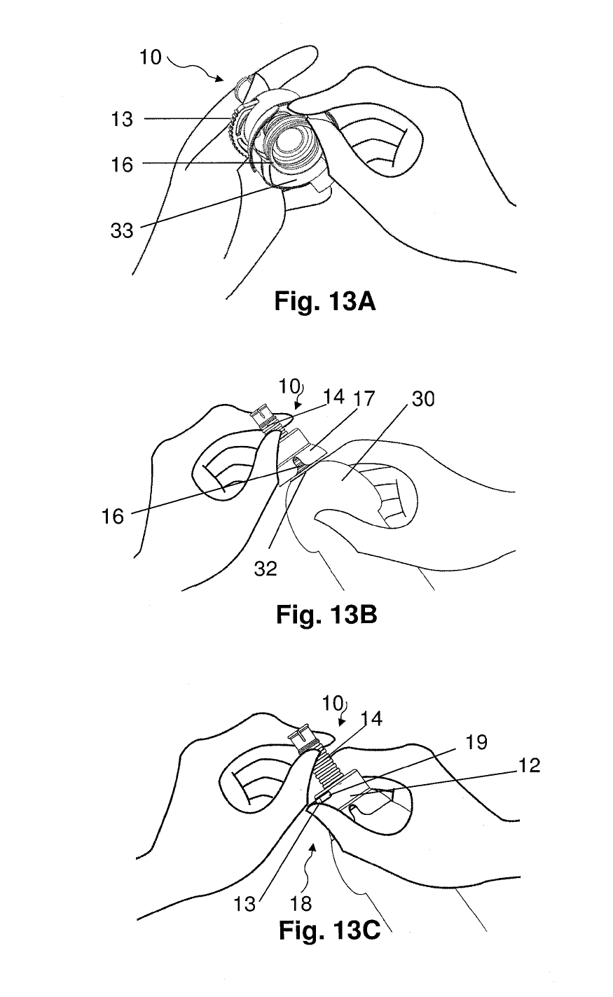

FIG. 7 is an isometric from-above-and-side view of an illustration of an assembly of components that construct a second embodiment of an external urinary incontinence treatment device of the present invention having a tube ratchet-locking system, in a configuration for the treatment of women.

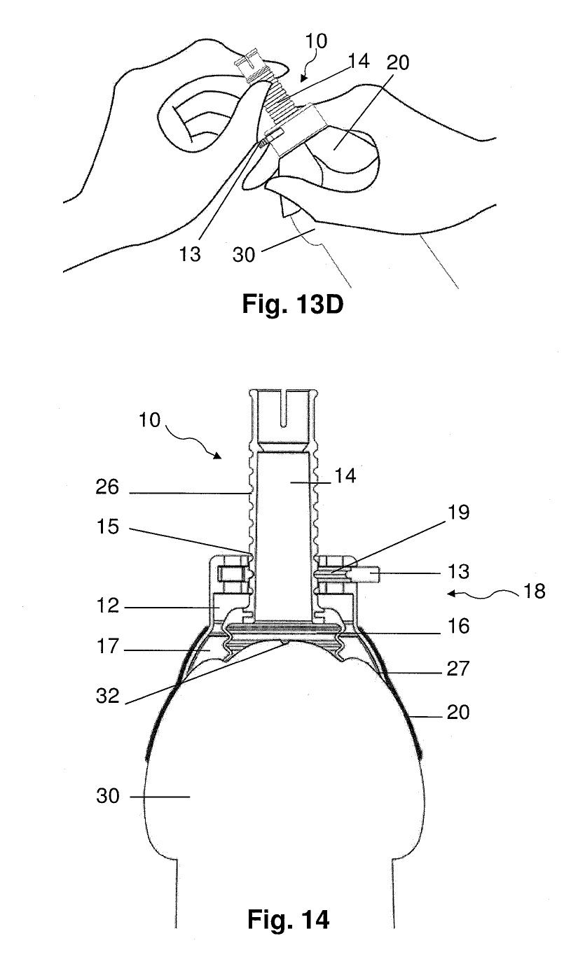

FIG. 8 is an isometric from-above-and-side view of an illustration of the embodiment of the external urinary incontinence treatment device shown in FIG. 7, in a partial assembled configuration.

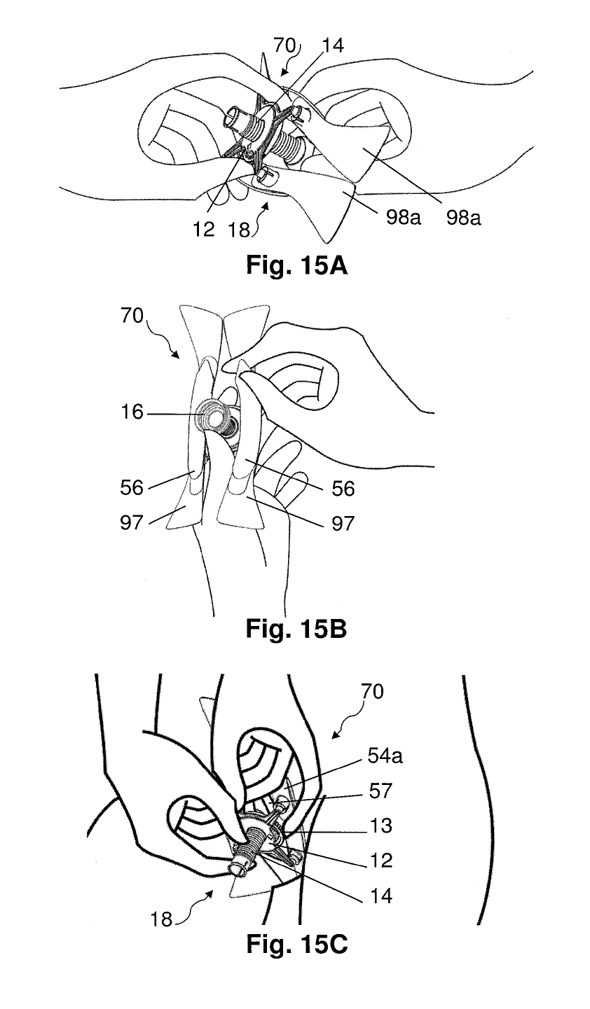

FIG. 9 is an isometric from-above-and-side view of an illustration of an assembly of components that construct a third embodiment of an external urinary incontinence treatment device of the present invention having a tube screw-locking system, in a configuration for the treatment of men.

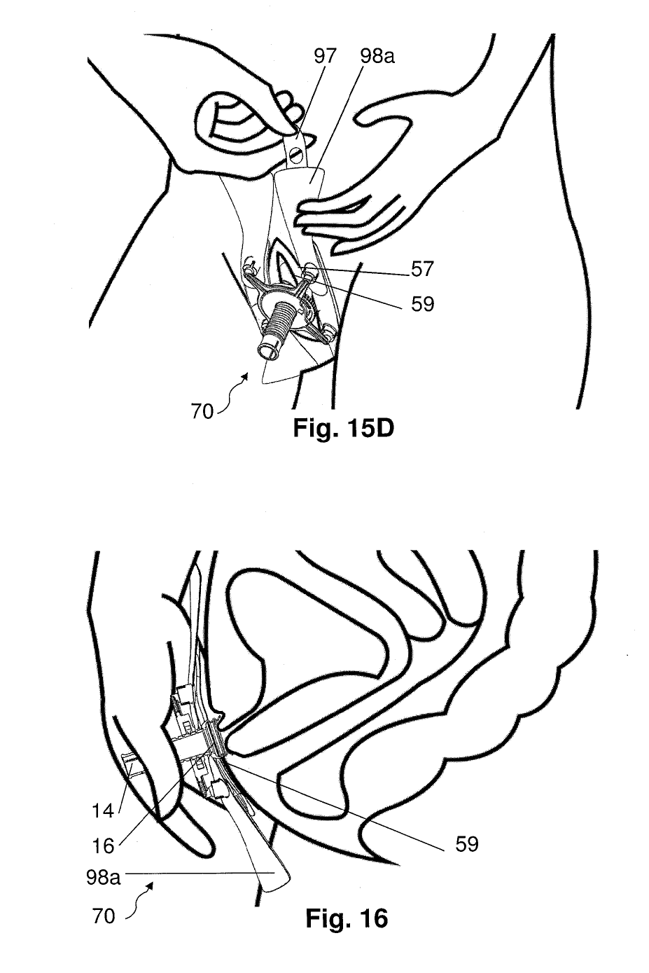

FIG. 10 is an isometric from-above-and-side view of an illustration of the embodiment of the external urinary incontinence treatment device shown in FIG. 9, in an assembled configuration.

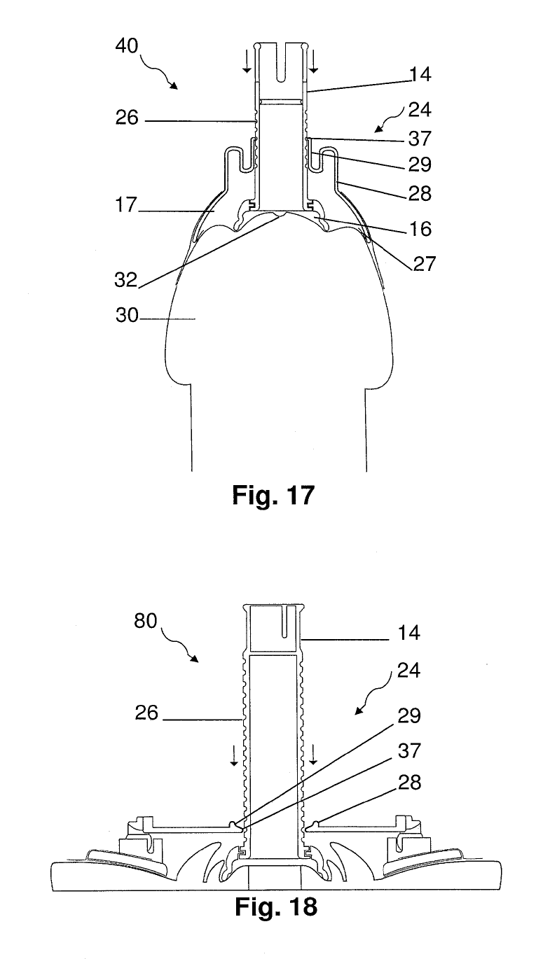

FIG. 11 is an isometric from-above-and-side view of an illustration of an assembly of components that construct a third embodiment of an external urinary incontinence treatment device of the present invention having a tube screw-locking system, in a configuration for the treatment of women.

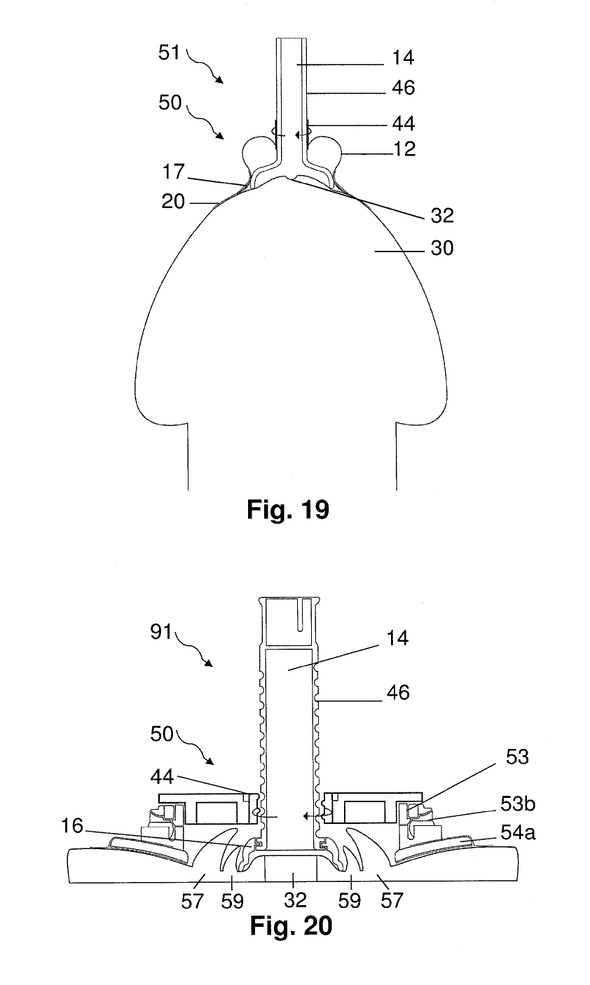

FIG. 12 is an isometric from-above-and-side view of an illustration of the embodiment of the external urinary incontinence treatment device shown in FIG. 11, in a partial assembled configuration.

FIG. 13A to FIG. 13D are illustrations of consecutive stages of deploying the embodiment of the external urinary incontinence treatment device of the present invention shown in FIG. 1 and FIG. 2.

FIG. 14 is a cross cut illustration of the embodiment of the external urinary incontinence treatment device of the present invention, with a clip-locking system, shown in FIG. 1 and FIG. 2, deployed in a treated patient.

FIG. 15A to FIG. 15D are illustrations of consecutive stages of deploying the embodiment of the external urinary incontinence treatment device of the present invention, with a clip-locking system, shown in FIG. 3 and FIG. 4.

FIG. 16 is a cross cut illustration of the embodiment of the external urinary incontinence treatment device of the present invention shown in FIG. 3 and FIG. 4, deployed in a treated patient. The figure illustrates the final deployment stage shown in FIG. 15A to FIG. 15D.

FIG. 17 is a cross cut illustration of the embodiment of the external urinary incontinence treatment device of the present invention, with a ratchet-locking system, shown in FIG. 5 and FIG. 6, deployed in a treated patient.

FIG. 18 is a cross cut illustration of the embodiment of the external urinary incontinence treatment device of the present invention, with a ratchet-locking system, shown in FIG. 7 and FIG. 8, deployed in a treated patient.

FIG. 19 is a cross cut illustration of the embodiment of the external urinary incontinence treatment device of the present invention, with a screw-locking system, shown in FIG. 9 and FIG. 10, deployed in a treated patient.

FIG. 20 is a cross cut illustration of the embodiment of the external urinary incontinence treatment device of the present invention, with a screw-locking system, shown in FIG. 11 and FIG. 12, deployed in a treated patient.

### DETAILED DESCRIPTION OF PREFERRED EMBODIMENTS

Three embodiments of an external urinary incontinence treatment device of the present invention are presently presented. Each of the embodiments is presented in a configuration for the treatment of both men (human males) and women (human females). In describing the embodiments the terms “human male” and “human female” are referred to interchangeably in the text that follows as: HM and HF, respectively.

The external urinary incontinence treatment device of the present invention is composed of: a receiving-component (16), a tube (14) that communicates freely with the receiving-component, a RC-supporting-element (12), a tube locking system connected to, or alternatively, integrated with, or alternatively is a part of, the RC-supporting-element. The locking systems presented in the text are: a clip-locking system (18), a ratchet-locking system (24) and a screw-locking system (50). The locking system is not limited to being one of the three presented mechanisms, and can have other configurations. The external urinary incontinence treatment device of the present invention also includes genital region connection component, designated: (17) for the HM configuration of the external urinary incontinence treatment device of the present invention and (54) for the HF configuration. In addition, the external urinary incontinence treatment device includes a thin adhesive film anchoring elements, designated (20) and (27) for the HM configuration, (52) and (98) for the HF configuration. The thin adhesive film anchoring elements are reversibly connected and covered by film protection components, designated (22) and (33) for HM configuration and (56) and (97) for the HF configuration.

Receiving component (16) typically has a dome-shape with a hole at its center, to which tube (14) connects. Alternatively, instead of two structural components, receiving component (16) and tube (14) can be produced as a component having a single entity. The circumference rim of the wall of the dome of receiving component (16) is designed to reversibly connect to the skin surrounding the urethras orifice of a treated patient in a urine leak-free connection when pressured against the skin.

Both receiving-component (16) and tube (14), are made of a semi-rigid material such as, but not limited to, various plastic materials, latex, nylon and rubber. Tube (14) is rigid, or semi rigid, at its proximal portion and is flexible at its distal portion. The distal end portion is typically designed to connect to a tube that leads to a urine collection bag and may have a curved configuration.

RC-supporting-element (12) which can have various configurations, is typically made of a semi-rigid material such as, but not limited to, various plastic materials, latex, nylon and rubber, enables the vertical motion of tube (14) (in the distal and proximal directions). The tube locking system component fixates the motion of tube (14) in the desired placement in the RC-supporting-element (12).

The thin adhesive film anchoring element is typically made of a thin adhesive film that either has a sticky surface on its proximal side and a plain, “not-sticky” surface on its distal side or a thin adhesive film that has a sticky surface on both sides of the film. Thin adhesive film anchoring elements (20) and (98) have a single sticky surface while thin adhesive film anchoring elements (27) and (52) have both sides “sicky”. An example of a single sticky surface thin adhesive film product that may be used is “Tegaderm”, produced by the 3M company (see websites: http://www.3m.com and http://solutions.3m.com/wps/portal/3M/en_US/3MSWC/Skin-Wound-Care/BrandsDirectory/Tegaderm/).

The film protection components are typically made of flexible material such as, but not limited to, paper, silicon, rubber thin sheet. The film protection components maintain the stickiness of the thin adhesive film of the thin adhesive film anchoring elements when the external urinary incontinence treatment device of the present invention is in storage. When the external urinary incontinence treatment device is deployed the film protection components are peeled off the thin adhesive film anchoring elements and discarded.

Typically, but not limited to, in the HM configuration of the external urinary incontinence treatment device of the present invention, RC-supporting-element (12) and the genital region connection component (17) are merged into a single entity with each portion of the single entity preforming a different function. Optionally, in the HF RC-supporting-element (12) and the genital region connection component (54) are merged into a single entity with each portion of the single entity preforming a different function.

Typically, in embodiments of the external urinary incontinence treatment device of the present invention, the thin adhesive film anchoring element has a HM configuration and a HF configuration.

In the HM configuration of the external incontinence treatment device, thin adhesive film anchoring element (20) is sheet of a thin adhesive film, shaped in a configuration that is, but is not limited to, a solid ring with or without a cut through the continuity of the ring or, alternatively, a set of leaves stemming from a thin ring. The spatial configuration of the thin adhesive film can be, but not limited to, a flat configuration or a dome-shaped configuration. Thin adhesive film anchoring element (20) is reversibly connected on its sticky, proximal side, to film protection component (22). Thin adhesive film anchoring element (20) with film protection component (22) are maintained separate and connect to the other components of the external incontinence treatment device when the device is deployed. Thin adhesive film anchoring element (27) is composed of at least one single sheet of a thin adhesive film component that is connected and covers the proximal portion of the inner wall of genital region connection component (17). Thin adhesive film anchoring element (27) may be, but is not limited to being, in a continuous or, alternatively, in a segmented ring configuration. The thin adhesive film of component (27) is connected to the inner wall with the distal sticky side of the film, thus, forming a sticky ring with the sticky side of the film around the proximal portion of genital region connection component (17). Thin adhesive film anchoring element (27) is reversibly connected on its sticky, proximal side, to film protection component (33). Film protection components (22) and (33) are peeled from thin adhesive film anchoring elements (20) and (27) when the external urinary incontinence treatment device is deployed.

In the HF configuration of the external incontinence treatment device, genital region connection component (54) is composed of two elongated sub-components (54a). The sub-component units of genital region connection component (54) are each connected to and covered by a sub-element (52a) of thin adhesive film anchoring element (52). Sub-elements (52a) are connected in their distal sticky side to the sub-components of genital region connection components (54a) and are reversibly connected on in their sticky, proximal side, to film protection components (56). Thin adhesive film anchoring element (98) is composed of four elongated sub-elements (98a). Each of the four sub-element (98a) of thin adhesive film anchoring element (98) is composed of a sheet of flexible material such as, but not limited to, paper, silicon, rubber thin sheet. which is reversibly connected to and covered by, on its proximal sticky side by a thin protection film sub-component (97a). The four sub-components (98a) comprise film protection component (97). Each of the two the sub-components of genital region connection component (54a) has connected in its distal side and protruding from it, two sub-elements (98a). Each of the sub elements is connected to the an edge of the elongated sub-component (54a), to element (53b), which is part of the construction of sub-component (54a). Film protection components (56) and (97) are peeled from thin adhesive film anchoring elements (52) and (98) when the external urinary incontinence treatment device is deployed.

Reference is presently made to three locking systems of the external urinary incontinence treatment device of the present invention, each activated and deactivated by a different mechanism: a clip-locking system (18), a ratchet-locking system (24) and a screw-locking system (50).

In the clip-locking system (18), illustrated in FIGS. 1 to 4, 13C and 15C, tube (14) has parallel circumferential groves (26) along the external side of the tube. Tube (14) is inserted into hole (15) that runs through the RC-supporting-element (12). Tube (14) is restricted by RC-supporting-element (12) to move vertically, in the distal and proximal directions. A slit (19) at the side of RC-supporting-element (12) has clip ring component (13) inserted into it. Clip ring component (13) is elliptical-ring structure, made of, but not limited to, a rigid plastic material, with a protruding thin-ring (35) in the inner wall of the clip component. Clip component (13) is free to slide horizontally within the slit (19) while encircling tube (14). The movement of clip (13) is caused by pressing on the clip. When clip (13) is not is not pressed it automatically returns to its placement in the slit (19), activating clip-locking system (18). Thus, when pressing and then releasing the pressing on clip (13), the configuration of clip-locking system (18) is transformed from being inactivated to activated. When clip ring component (13) is slid in slit (19) so that the inner-ring (35) of the clip (13) enter between the groves (26) of tube (14) and interlocks with them, tube (14) is fixated and can no longer move within RC-supporting-element (12). When clip ring component (13) is slid in the opposite direction, freeing the interlocking between groves (26) and the inner-ring (35), tube (14) resumes the ability to move vertically within RC-supporting-element (12).

In the ratchet-locking system (24), illustrated in FIGS. 5, 6, 7, 8, 17 and 18, tube (14) has parallel circumferential groves (26) along the external side of the tube. Tube (14) is inserted in hole (15) in RC-supporting-element (12). Hole (15) is surrounded by flexible support bars ((28) and optionally (29)) that have at their end protruding teeth (37). The protruding teeth (37) in RC-supporting-element (12) reversibly insert into groves (26) of tube (14) in a configuration that forms a ratchet interlock. The ratchet interlock restricts movement of tube (14) in the proximal direction while preventing the reverse movement, in the distal direction. When the receiving component (16) which is connected to tube (14) encounters the rigid surface of the skin surrounding the urethral orifice in the course of the proximal direction movement of tube (14), tube (14) is adjusted and fixated in place and can not move in the distal direction. By slightly bending and changing the spatial configuration of the support bars ((28) and (29)) in RC-supporting-element (12) with the fingers of a hand, the teeth (37) of the ratchet are withdrawn from the groves (26) and tube (14) is able to move vertically, in the distal direction.

In the screw-locking system (50), illustrated in FIGS. 9, 10, 11, 12, 19 and 20, tube (14) has a screw-tread (46) along the external side of the tube. A tube-supporting-ring (44), positioned in the center of RC-supporting element (12), has a screw-thread in its internal side and screws over the screw thread (46) of tube (14). By turning the tube-supporting-ring (44) around the tube, tube (14) is gradually moved by the screw-thread in the proximal direction. By reversing the turning direction of tube-supporting-ring (44), the tube is withdrawn in the distal direction. When tube-supporting-ring (44) is not turned, tube (14) us fixated in place in RC-supporting element (12).

Optionally, the urine receiving component (16), the a tube (14) and tube locking system of choice of external urinary incontinence treatment device of the present invention can have a configuration of a single entity component. Reference is presently made to the procedure of deploying the external urinary incontinence treatment device of the present invention.

In preparing for deployment, for the HF configuration of the device the procedure starts with connecting genital region connection component (54) with the RC-supporting-element (12). In both the HM and HF configuration of the device, the deployment of the device starts by removing the film protection component (33) for HM and (56) for HF, from thin adhesive film anchoring element (27) HM and (52) for HF. In the next stage the external urinary incontinence treatment device is positioned over the genital region and receiving component (16) is placed over the urethra orifice with the rim of component (16) right above or slightly touching the surrounding the orifice. In the next stage genital region connection component, (17) for HM (54) for HF, is connected to the genital region by thin adhesive film anchoring element (27) HM and (52) for HF. In the following stage, the film protection component (22) for HM and (56) for HF, is removed from thin adhesive film anchoring element (20) HM and (98) for HF, and the film anchoring element is connected to the genital region. In the next stage, tube (14), which is inserted in RC-supporting-element (12) is moved (pressed) in the proximal direction, towards the treated patient. In moving tube (14), receiving-component (16), which is connected to tube (14), connects with the skin in the circumference of the urethral orifice of the treated patient. The opened side of the dome the receiving-component is adjusted and fastened to the skin in urine leak-free connection. In the next stage, when the desired fastness to the skin around the urethral orifice is achieved, tube (14) is reversibly fixated in place by the locking system ((18) or (24) or (50), depending on the locking system utilized. The fixating of tube (14) can easily be undone and again reintroduced, thus allowing for the rapid and frequent adjustment, fastening and fixation of the receiving component so as to minimize the inconvenience of the treated patient yet maintaining a urine leak-free connection between the genital region and the external urinary incontinence treatment device.

Reference is presently made to the figures, which illustrate embodiments of the present invention.

FIG. 1 is an isometric from-above-and-side view of an illustration of an assembly of components that construct an embodiment of an external urinary incontinence treatment device (10) of the present invention having a tube clip-locking system, in a configuration for the treatment of men. Listed from top to bottom: thin (cut through, designated (20a)) adhesive film anchoring element (20), film protection component (22), RC supporting element (12) with a hole (15) at its center and a connected dome shaped genital region connection component (17) which is the proximal portion of element (12), a clip ring component (13), tube (14), receiving-component (16), a second thin adhesive film anchoring element (27) and a second film protection component (33).

FIG. 2 is an isometric from-above-and-side view illustration of the embodiment of the external HM urinary incontinence treatment device (10) shown in FIG. 1, in an assembled configuration, without showing the adhesive film anchoring elements. Clip ring component (13) is shown inserted into slit (19) on the side of RC supporting element (12). Tube (14) is shown inserted in hole (15) in RC supporting element (12).

FIG. 3 is an isometric from-above-and-side view of an illustration of an assembly of components that construct an embodiment of an external urinary incontinence treatment device (70) of the present invention having a tube clip-locking system, in a configuration for the treatment of women. Listed from top to bottom: RC supporting element (12) with a hole (15) at its center, clip ring component (13), tube (14), receiving-component (16), thin adhesive film anchoring element (98), film protection components (97), genital region connection component (54) which is divided to two sub-components (54a). a second thin adhesive film anchoring element (52) which is divided to two sub-components (52a), and a second film protection component (56), which is divided to two sub-components (56a).

FIG. 4 is an isometric from-above-and-side view illustration of the embodiment of the external HF urinary incontinence treatment device (70) shown in FIG. 3, in a partial assembled configuration, shown without the adhesive film anchoring elements and with only one connection element 54a. Clip ring component (13) is shown inserted into slit (19) on the side of RC supporting element (12). Tube (14) is shown inserted in hole (15) in RC supporting element (12). RC supporting element (12) is connected to four bar-legs (53) that are connected and are an integral part of RC supporting element (12). Bar-legs (53) connect RC supporting element (12) to genital region connection component (54). Alternatively, bar-legs (53) are not an integral part RC supporting element (12) and are separate components made of, but not limited to, plastic material, metal material, spring, and rigid-wire. Optionally, the sub-components of genital region connection component (54a) with the sub-elements (52a) of thin adhesive film anchoring element (52) are kept separate from RC-supporting-element (12). Sub components (54a) are connected to RC-supporting-element (12) via bar-legs (53) only when the HF configuration of the external incontinence treatment device is deployed. Bar-legs (53) are connected to sub-elements (54a) via a spherical connecting element designated (53a) at the end of bar-legs (53), which reversibly connects to connecting element (53b) which is a socket configuration and is a part of the construction of sub-components of genital region connection component (54a).

FIG. 5 is an isometric from-above-and-side view of an illustration of an assembly of components that construct a second embodiment of an external urinary incontinence treatment device (40) of the present invention having a tube ratchet-locking system, in a configuration for the treatment of men. Listed from top to bottom: thin adhesive film (cut through (20a) anchoring element (20), film protection component (22), RC supporting element (12) with a hole (15) at its center and a connected dome shaped genital region connection component (17) which is the proximal portion of element (12), tube (14), receiving-component (16), a second thin (full ring) shaped adhesive film anchoring element (27) and a second (full ring) film protection component (33).

FIG. 6 is an isometric from-above-and-side view illustration of the embodiment of the external HM urinary incontinence treatment device (40) shown in FIG. 5, in an assembled configuration, without the adhesive film anchoring elements. Tube (14) is shown inserted in hole (15) in RC supporting element (12). The ratchet-locking system (24), shown in the figure, is composed of ring of flexible support-bars (28) that surround hole (15) which with flexible U shaped support components (29) connected and extended from their distal top-side. A protruding tooth (37) at the free end facing tube (14) of U shaped components (29) interlocks with the groves (26) in tube (14). The U shaped components (29) are positioned in a slanted angle to the flat-bars. Optionally, the components connected to flat bars (28), designated (29), can be at a straight angle to (28) and can have various configurations, such as but not limited to, an S form.

FIG. 7 is an isometric from-above-and-side view of an illustration of an assembly of components that construct a second embodiment of an external urinary incontinence treatment device (80) of the present invention having a tube ratchet-locking system (24), in a configuration for the treatment of women. Listed from top to bottom: RC supporting element (12) with a hole (15) at its center, tube (14), receiving-component (16), thin adhesive film anchoring element (98), film protection components (97), genital region connection component (54), a second thin adhesive film anchoring element (52) and a second film protection component (56). The structure of the components: thin adhesive film anchoring element (98), film protection components (97), genital region connection component (54), second thin adhesive film anchoring element (52) and second film protection component (56) in device (80), illustrated in FIG. 7 and FIG. 8, are identical to the components of device (70) and where described in detail in explaining FIG. 3 and FIG. 4.

FIG. 8 is an isometric from-above-and-side view illustration of the embodiment of the external HF urinary incontinence treatment device (80) shown in FIG. 7, in a partial assembled configuration without the adhesive film anchoring elements, shown without the adhesive film anchoring elements and with only one connection element (54a). The ratchet-locking system (24), shown in the figure, is similar to ratchet-locking system (24) illustrated and explained in FIG. 5 and FIG. 6. The ratchet-locking system (24), which is connected to RC supporting element (12), is composed of ring of flexible support-bars (28) that surround hole (15) with an optional addition of connected flexible U shaped support components (29). To support bars (28) or, optionally to support components (29), connected and extended from their distal top-side are protruding teeth (37). Teeth (37) face tube (14) and interlocks with the groves (26) in tube (14).

FIG. 9 is an isometric from-above-and-side view of an illustration of an assembly of components that construct a third embodiment of an external urinary incontinence treatment device (51) of the present invention having a tube screw-locking system (50), in a configuration for the treatment of men. Listed from top to bottom: thin (full ring, dome shaped) adhesive film anchoring element (20), (domed shaped, full ring) film protection component (22), tube-supporting-screw-ring component (44) with screw thread grooves in the internal part of the ring, RC supporting element (12) with a hole (15) at its center, tube (14) with a screw thread grooves (46) on the circumference of tube connected to receiving-component (16), a second thin (fragmented ring) adhesive film anchoring element (27) and a second (fragmented ring) film protection component (33). RC-supporting-element (12) is composed of a ring (11) and is connected to flexible curved bands (31) that form a genital region connection component (17).

FIG. 10 is an isometric from-above-and-side view of an illustration of the embodiment of the external urinary incontinence treatment device shown in FIG. 9, in an assembled configuration, without the adhesive film anchoring elements. In the figure tube (14) is shown inserted into tube-supporting-screw-ring component (44). Tube-supporting-screw-ring component (44) is positioned in hole (15) in ring (11) of RC-supporting-element (12) and is free to turn within ring (11). Turning tube-supporting-screw-ring component (44) tube (14) is moved vertically in the distal and proximal direction, depending on the direction of the turning of component (44). When tube-supporting-screw-ring component (44) not turned tube (14) is fixated in place.

FIG. 11 is an isometric from-above-and-side view of an illustration of an assembly of components that construct a third embodiment of an external urinary incontinence treatment device (91) of the present invention having a tube screw-locking system (50), in a configuration for the treatment of woman. Listed from top to bottom: turning tube-supporting-screw-ring component (44), RC supporting element (12) with a hole (15) at its center, tube (14) with a screw thread grooves (46) on the circumference of tube, receiving-component (16), thin adhesive film anchoring element (98), film protection components (97), genital region connection component (54), a second thin adhesive film anchoring element (52) and a second film protection component (56). The sub-structure of the components: thin adhesive film anchoring element (98), film protection components (97), genital region connection component (54), second thin adhesive film anchoring element (52) and second film protection component (56) in device (91), illustrated in FIG. 11 and FIG. 12, are identical to the components of device (70) and where described in detail in explaining FIG. 3 and FIG. 4.

FIG. 12 is an isometric from-above-and-side view of an illustration of the embodiment of the external urinary incontinence treatment device (91) shown in FIG. 11, in a partial assembled configuration, shown without the adhesive film anchoring elements and with only one connection element (54a). In the figure tube (14) is shown inserted into tube-supporting-screw-ring component (44). Tube-supporting-screw-ring component (44) is positioned in hole (15) in ring (11) of RC-supporting-element (12) and is free to turn within ring (11). Tube-supporting-screw-ring component (44) is positioned in hole (15) in ring (11) of RC-supporting-element (12) and is free to turn within ring (11). When turning tube-supporting-screw-ring component (44), tube (14) is moved vertically in the distal and proximal direction, depending on the direction of the turning of component (44). When tube-supporting-screw-ring component (44) is not turned, tube (14) is fixated in place.

FIG. 13A to FIG. 13D are illustrations of consecutive stages of deploying the embodiment of the external urinary incontinence treatment device (10) of the present invention shown in FIG. 1 and FIG. 2. The illustrated deployment stages in FIGS. 13A to 13D are identical for the deployment of the embodiments of the external urinary incontinence HM treatment device designated: (40) and (51) and shown in FIGS. 5-6 and FIGS. 9-10, respectively, with the exception that the tube locking system in (40) is a ratchet-locking system and in (51) a screw-locking system. Each tube locking system is activated and deactivated by a different locking procedure.

FIG. 13A illustrates the first stage of deploying the external urinary incontinence HM treatment device (10). Device (10) is held in one hand and with the other hand film protection component (33) is peeled off from thin adhesive film anchoring element (27), thus, exposing the surface of the sticky side of the component.

FIG. 13B illustrates the second stage of deploying the external urinary incontinence HM treatment device (10). Device (10) is positioned over the glans penis (30) of the treated patient with the receiving component (16) over the urethra orifice and the rim of the component slightly touching or very close to the surrounding of the orifice (32). Clip-locking system (18) is activated and tube (14) is not free to move vertically in the RC-supporting-element (12).

FIG. 13C illustrates the third stage of deploying the external urinary incontinence HM treatment device (10). With one hand tube (14) is held. With the other hand the clip-locking system (18) and the RC supporting element (12) are held and device (10) is pressed in the proximal direction till the genital region connection component (17) via thin adhesive film anchoring element (27), connects to the glans penis (30).

FIG. 13D illustrates the fourth stage of deploying the external urinary incontinence HM treatment device (10). With device (10) connected the glans penis (30). Using both hands, film protection component (22) is peeled off from thin adhesive film anchoring element (20). Film (20) is wrapped around genital region connection component (17) and connects the component with the glans penis (30). The illustration shows device (10) fixated in place with film (20) wrapped over the glans penis (30). With one hand the user now moves/presses the clip ring component (13) in clip-locking system (18) which releases the tube (14) from its locked position. The now free to-move tube (14) is pushed in the proximal direction, thus by pressing the pressure forms a leak free contact between the rim of receiving component (16) and the skin surrounding the urethral orifice (32). The receiving component (16) is adjusted and fastened to the glans penis (30). With the cooperation of the patient (when treated by a care giver), the most convenient combination (“compromise”) of having the receiving component (16) connected to the skin surrounding the urethral orifice (32) in a urine leak-tight connection and the minimal connection pressure and the receiving component (16) placement of connection applied on the glans penis, is obtained. When the most convenient combination is obtained, the user will press/release clip (13), and by so activate the locking in clip-locking system (18), thus, fixating in place tube (14). Fixating tube (14) fixates receiving component (16). By again moving/pressing clip ring component (13) clip-locking system (18) frees tube (14) to move, thus enabling the readjustment of the connection of receiving component (16) to the skin surrounding the urethral orifice (32). When the readjusting is made, tube (14) is relocked in place by press/releasing clip (13) and activating the clip-locking system (18).

FIG. 14 is a cross cut illustration of external urinary incontinence HM treatment device (10) with a clip-locking system (18) deployed in a treated patient. The illustration shows the positioning of the components that construct device (10), shown in FIG. 1, when device (10) is deployed in a treated patient. The figure illustrates the wrapping around of connection component (17) by adhesive film anchoring element (20) and the connection to the glans penis (30) of the treated patient.

FIG. 15A to FIG. 15D are illustrations of consecutive stages of deploying the embodiment of the external urinary incontinence treatment device (70) of the present invention shown in FIG. 3 and FIG. 4. FIG. 16 is the final deployment stage of the stages illustrated in FIG. 15A to FIG. 15D. The illustrated deployment stages in FIGS. 13A to 13D are identical for the deployment of the embodiments of the external urinary incontinence HF treatment device designated: (80) and (91) and shown in FIGS. 7-8 and 11-12, respectively, with the exception that the tube locking system in (80) is a ratchet-locking system and in (91) the tube locking system is a screw-locking system.

FIG. 15A illustrates the first stage of deploying the external urinary incontinence HF treatment device (70). Tube (14) with connected receiving component (16) is inserted into the RC supporting element (12). In device (70) the RC supporting element (12) is connected to the genital region connection component (54) which is connected to the RC supporting element (12) via supporting legs (53).

FIG. 15B illustrates the second stage of deploying the external urinary incontinence HF treatment device (70). The film protection sub-components (56a) are peeled off from thin adhesive film of the two sub anchoring elements (52a), thus exposing the surface of the sticky side of the (52a) sub elements.

FIG. 15C illustrates the third stage of deploying the external urinary incontinence HF treatment device (70). The patient holds tube (14) with one hand and with the other hand opens the lips of the labia minora (59).

With the lips opened, the patient positions the receiving component (16) over the surrounding area of the urethral orifice (32). After positioning the receiving component (16), with one hand the user holds tube (14) stably so that that the rim of receiving component (16) surrounds the urethral orifice (32) slightly touching or very close to the surrounding of the orifice (32). With the other hand the user presses the RC supporting element (12) in the proximal direction with the two sub anchoring elements (52a) on the two external sides of the labia majora (57). RC-supporting element (12) is pressed for some seconds to establish a stable reversible connection between the skin of the treated patient and sub anchoring elements (52) via thin adhesive film of the two sub anchoring element (52a). After forming the connection the user has both her hands free for the next stage.

FIG. 15D illustrates the fourth stage of deploying the external urinary incontinence HF treatment device (70). With the receiving component (16) over and slightly touching or very close to the surrounding of the urethral orifice (32), the RC-supporting-element (12) stably connected to the genital region of the treated patient, and tube (14) locked in place by the clip-locking system (18), using both hand film protection sub-component (97a) is peeled off from a thin adhesive film anchoring sub elements (98a). The sticky side of the adhesive film anchoring sub-elements (98a) are connected to the genital region of the patient, towards the belly and between the legs of the treated patient.

FIG. 16 is a cross cut illustration of the embodiment of the external HF urinary incontinence treatment device (70) in the process of being deployed in a treated patient. The figure illustrates the fifth stage of deploying the external urinary incontinence HF treatment device (70). Using one, hand clip ring component (13) is pressed/moved, thus deactivating clip-locking system (18) and freeing tube (14) to move vertically within RC supporting element (12). With the locking system deactivated, the hand keeps holding the RC-supporting-element (12). With RC supporting element (12) stably held in place, using the second hand, tube (14) is pushed in the proximal direction thus forming contact between the rim of receiving component (16) and the skin surrounding the urethral orifice (32). With the cooperation of the patient or according to the decision of the of the patient deploying device (70), the most convenient combination (“compromise”) of having the receiving component (16) connected to the skin surrounding the urethral orifice (32) in a urine leak-tight connection and the minimal pressure required for the urine leak-tight connection is obtained. When the most convenient combination is obtained, with the hand holding RC-supporting-element (12) clip ring component (13) is moved/released, thus fixating in place tube (14), causing receiving component (16) to be fixated. By again moving/pressing clip ring component (13), clip-locking system (18) frees tube (14) to move, thus enabling the readjustment of the connection of receiving component (16) to the skin surrounding the urethral orifice (32). When the readjusting is made, tube (14) is relocked in place by releasing clip (13) and reactivating the locking of clip-locking system (18).

FIG. 17 is a cross cut illustration of the embodiment of the external urinary incontinence HM treatment device (40), with a ratchet-locking system (24) shown in FIG. 6, shown in deployment in a treated patient. The illustration shows the positioning of the components that construct device (40), shown in FIG. 5, when device (40) is deployed in a treated patient. The procedure of deployment external urinary incontinence HM treatment device (40) is identical with the deployment of the external urinary incontinence treatment device (10), described in FIG. 13A to FIG. 13D, except for the fixating of tube (14). The figure illustrated the mechanism of operating the ratchet-locking system (24): Tube (14) is shown moved in the proximal direction (indicated by the arrows). Groves (26) on tube (14) are shown interlocked with protruding tooth (37). When no pressure is applied tube (14) is fixated in place. With tube (14) fixated, the rim of receiving component (16) is fastened and fixated to the skin surrounding the urethral orifice (32).

FIG. 18 is a cross cut illustration of the embodiment of the external urinary incontinence HF treatment device (80), with a ratchet-locking system (24), shown in FIG. 8. The illustration shows the positioning of the components that construct device (80), shown in FIG. 7, when device (80) is deployed in a treated patient. The procedure of deployment external urinary incontinence treatment device (80) is identical with the deployment of the external urinary incontinence treatment device (70), described in FIG. 15A to FIG. 16, except for the fixating of tube (14). The mechanism of operating the ratchet-locking system (24) was previously explained in FIG. 17).

FIG. 19 is a cross cut illustration of the embodiment of the external urinary incontinence HM treatment device (51), with a screw-locking system (50) shown in FIG. 10, shown in the illustration in deployment in a treated patient. The illustration shows the positioning of the components that construct device (51), shown in FIG. 9, when device (51) is deployed in a treated patient. The procedure of deployment external urinary incontinence HM treatment device (51) is identical with the deployment of the external urinary incontinence treatment device (10), described in FIG. 13A to FIG. 13D except for the fixating of tube (14). The figure illustrated the mechanism of operating the screw-locking system (50): Tube (14) is moved in the proximal or distal direction in accordance with the direction of turning tube supporting screw ring (44) which turns over screw-tread (46) on tube (14), as indicated by the arrow. When receiving component (16) is adjusted and fastened to the skin surrounding the urethral orifice (32), tube supporting screw ring (44) is no longer turned, thus, no movement pressure is applied and tube (14) is fixated in place.

FIG. 20 is a cross cut illustration of the embodiment of the external urinary incontinence HF treatment device (91), with a screw-locking system (50), shown in FIG. 12. The illustration shows the positioning of the components that construct device (91), shown in FIG. 11, when device (91) is deployed in a treated patient. The procedure of deployment external urinary incontinence treatment device (91) is identical with the deployment of the external urinary incontinence treatment device (70), described in FIG. 15A to FIG. 15D except for the fixating of tube (14). The mechanism of operating the screw-locking system (50) was previously explained, in FIG. 19.

It should also be clear that a person skilled in the art, after reading the present specification could make adjustments or amendments to the attached Fig. and above described embodiments that would still be covered by the present invention.

I claim:

1) An external urinary incontinence treatment device for a human patient
comprising:
a urine receiving component,
a tube,
a receiving component supporting element,
a tube locking system,
a genital region connection component,
a genital region anchoring element,
wherein, said tube connects and communicates freely with said urine receiving-component,
wherein, said tube is inserted through said receiving component supporting element and communicates with said tube locking system,
wherein, said tube is able to move vertically in said receiving component supporting element when said tube locking system is deactivated and said tube is fixated in its movement at a desired position along the length of said tube when said tube locking system is activated,
wherein, said receiving component supporting element is connected to said genital region connection component and with said tube locking system,
wherein, said genital region connection component is connected to said genital region anchoring element,
wherein, said genital region anchoring element is reversibly connected to the skin of the genital region of said patient,
wherein, said urine receiving-component is connected to the skin surrounding the urethral orifice of the treated patient when said tube is moved towards the genital region of said patient,
wherein, said urine receiving component applies pressure to the skin surface surrounding the urethral orifice of said patient with continued movement of said urine receiving component towards the genital region of said patient.

2) The tube and urine receiving component of external urinary incontinence treatment device of claim 1, wherein, the tube and the urine receiving component have a single entity.

3) The urine receiving component of external urinary incontinence treatment device of claim 1, wherein, the urine receiving component has a dome configuration.

4) The urine receiving component, the a tube and tube locking system of external urinary incontinence treatment device of claim 1, wherein the urine receiving component, the tube and a tube locking system have a single entity component.

5) The receiving component supporting element and genital region anchoring element of external urinary incontinence treatment device of claim 1, wherein the receiving component supporting element and genital region anchoring element have a configuration of a single entity component.

6) The urine receiving component of external urinary incontinence treatment device of claim 1, wherein, pressure that is applied to the skin surface surrounding the urethral orifice by said urine receiving component is also applied to the genital region of said patient by said genital region connection component which is connected to said genital region anchoring element.

7) The tube locking system of external urinary incontinence treatment device of claim 1, wherein, the locking system is a clip-lock mechanism.

8) The tube locking system of external urinary incontinence treatment device of claim 1, wherein, the locking system is a ratchet mechanism.

9) The tube locking system of external urinary incontinence treatment device of claim 1, wherein, the locking system is a screw mechanism.

10) The tube locking system of external urinary incontinence treatment device of claim 1, wherein, the locking system is any mechanism that when activated fixates said tube from moving vertically in said receiving component supporting element and when deactivated enables said tube to move vertically in said receiving component supporting element.

11) The genital region anchoring element of external urinary incontinence treatment device of claim 1, wherein said genital region anchoring element is composed of a thin adhesive film.

12) The thin adhesive film of the genital region anchoring element of claim 10, wherein said thin adhesive film is reversibly connected to a film protection component.

13) Thin adhesive film of anchoring element of external urinary incontinence treatment device of claim 10, wherein the thin adhesive film is reversibly connected to genital region connection component of claim 1.

14) The genital region anchoring element of external urinary incontinence treatment device of claim 1, wherein said genital region anchoring element is composed of at least one structural element that reversibly connects said genital region connection component to the body of the treated patient.

15) The structural element of the genital region anchoring element of external urinary incontinence treatment device of claim 13, wherein the structural element has a strap configuration.

16) The structural element of the genital region anchoring element of external urinary incontinence treatment device of claim 13, wherein the structural element has a string configuration.

17) The structural element of the genital region anchoring element of external urinary incontinence treatment device of claim 12, wherein the structural element has a wire configuration.

18) The structural element of the genital region anchoring element of external urinary incontinence treatment device of claim 13, wherein the structural element has a spring configuration.

19) A method for operating an external urinary incontinence treatment device of a human patient,
the device comprising:
a urine receiving component that is connected to a tube and freely communicates with said tube,
said tube is connected to a receiving component supporting element and to a tube locking system,
said receiving component supporting element is connected to a genital region connection component,
said genital region connection component is connected to a genital region anchoring element,
wherein, said tube is able to move vertically in said receiving component supporting element when said tube locking system is deactivated and said tube is fixated in its movement at a desired position along the length of said tube when said tube locking system is activated,
wherein operating said device, said genital region anchoring element is reversibly connected to the skin of the genital region of said patient and said urine receiving-component is connected to the skin surrounding the urethral orifice of the treated patient when said tube is moved towards the genital region of said patient,
wherein, said urine receiving component applies pressure to the skin surface surrounding the urethral orifice of said patient with continued movement of said urine receiving component towards the genital region of said patient,
wherein, said tube is reversibly fixated in place by said tube locking system after urine receiving component is adjusted and fastened to the skin surrounding the urethral orifice of said patient in a urine leak free connection while causing said treated patient minimal inconvenience.
